(19) United States
(12) Patent Application Publication
(10) Pub. No.: US 2015/0051531 A1
(43) Pub. Date: Feb. 19, 2015

(54) DRESSING STRUCTURE

(71) Applicant: Tatung Company, Taipei (TW)

(72) Inventor: Cheng-Yang Lee, Taipei (TW)

(73) Assignee: Tatung Company, Taipei (TW)

(21) Appl. No.: 14/149,811

(22) Filed: Jan. 8, 2014

(30) Foreign Application Priority Data
Aug. 16, 2013 (TW) 102215493

(51) Int. Cl. A61F 13/02

(52) U.S. Cl. CPC A61F 13/0243
USPC 602/43

(57) ABSTRACT

A dressing structure comprising a composite substrate layer and a gel layer is provided. The composite substrate layer comprises a first substrate layer and a second substrate layer. The first substrate layer comprises a plurality of first pores. The second substrate layer comprises a plurality of second pores. The second substrate layer and the first substrate layer are flat and unwrinkled. The gel layer is at least disposed between the first substrate layer and the second substrate layer.

### CROSS-REFERENCE TO RELATED APPLICATION

This application claims the priority benefit of Taiwan application no. 102215493, filed on Aug. 16, 2013. The entirety of the above-mentioned patent application is hereby incorporated by reference herein and made a part of this specification.

### BACKGROUND OF THE INVENTION

1. Field of the Invention

The invention relates to a dressing structure, and more particularly, to a dressing structure having a composite substrate layer.

2. Description of Related Art

The fabrication process of a dressing generally includes the following steps. First, a primary material of the dressing is coated on a substrate layer to react. Then, the process is finished by cropping, posting, stamping, and packaging according to the desired size. In particular, since the primary material is usually a runny liquid and the substrate layer is a porous material, therefore, when coating the primary material, the primary material is embedded in the pores of the substrate layer to facilitate physical adhesion and polymerization at the same time in order to complete the fabrication of solid gels, such as hydrogel. However, the polymerization causes the loss of moisture inside the primary material and creates cohesion during a curing process of the primary material, which the substrate layer generates wrinkles due to the stress of pulling and squeezing. Since the wrinkles generated after the primary material is cured cannot be removed, dressing products have a poor appearance; when using it, there are defects, such as difficulties of being adhering evenly on the skin, discomfort, and so on.

### SUMMARY OF THE INVENTION

The invention provides a dressing structure, which enhances the stability of a dressing and improves the convenience and comfort in the use of the dressing.

Accordingly, the present invention is directed to a dressing structure. The dressing structure includes a composite substrate layer and a gel layer. The composite substrate layer includes a first substrate layer and a second substrate layer. The first substrate layer includes a plurality of first pores. The second substrate layer includes a plurality of second pores. The second substrate layer and the first substrate layer are flat and unwrinkled. The gel layer is at least disposed between the first substrate layer and the second substrate layer.

In an embodiment of the invention, the first substrate layer and the second substrate layer include porous membranes.

In an embodiment of the invention, the gel layer includes a hydrogel layer or a polymer layer.

In an embodiment of the invention, the gel layer is further located in the first pores of the first substrate layer.

In an embodiment of the invention, the first pores of the first substrate layer are filled up with the gel layer so that the gel layer is further disposed on a surface of the first substrate layer.

In an embodiment of the invention, a size of the second pores is smaller than a size of the first pores.

In an embodiment of the invention, a size of the second pores is equal to a size of the first pores.

In an embodiment of the invention, a size of the second pores is larger than a size of the first pores.

In an embodiment of the invention, a back layer is further included, which is disposed on a surface of the second substrate layer opposite to the gel layer.

In an embodiment of the invention, a barrier layer is further included, which is disposed between the back layer and the second substrate layer.

In an embodiment of the invention, the barrier layer includes a non-porous membrane.

In an embodiment of the invention, the barrier layer includes a lamination layer.

In an embodiment of the invention, a material of the barrier layer includes polyethylene, polypropylene, polyester, polycarbonate, polyurethanes, a derivative thereof, or a combination thereof.

In an embodiment of the invention, an adhesive layer is further included, which is disposed between the barrier layer and the second substrate layer.

In an embodiment of the invention, the adhesive layer includes a hot melt adhesive, a pressure sensitive adhesive or acrylic glue.

In an embodiment of the invention, a material of the first substrate layer and a material of the second substrate layer independently include cotton, hemp, or chemical synthetic fiber.

In an embodiment of the invention, a material of the chemical synthetic fiber includes polyethylene, polypropylene, polyester, polycarbonate, polyurethanes, a derivative thereof, or a combination thereof.

In an embodiment of the invention, the materials of the first substrate layer and the second substrate layer are the same.

In an embodiment of the invention, the materials of the first substrate layer and the second substrate layer are different.

Based on the above, the design of the composite substrate layer of the invention can avoid the defects, such as a poor product appearance, difficulties of being adhering evenly on the skin, discomfort, and so on, thereby enhancing the stability of the dressing and improves the convenience and comfort in the use of the dressing.

In order to make the aforementioned and other features and advantages of the invention more comprehensible, several embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic cross-sectional view of a dressing structure according to a first embodiment of the invention.

FIG. 2A through FIG. 2D are schematic perspective views of a fabrication process of the dressing structure shown in FIG. 1.

FIG. 3 is a schematic cross-sectional view of a dressing structure according to a second embodiment of the invention.

FIG. 4 is a schematic cross-sectional view of a dressing structure according to a third embodiment of the invention.

FIG. 5 is a schematic cross-sectional view of a dressing structure according to a fourth embodiment of the invention.

### DESCRIPTION OF EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

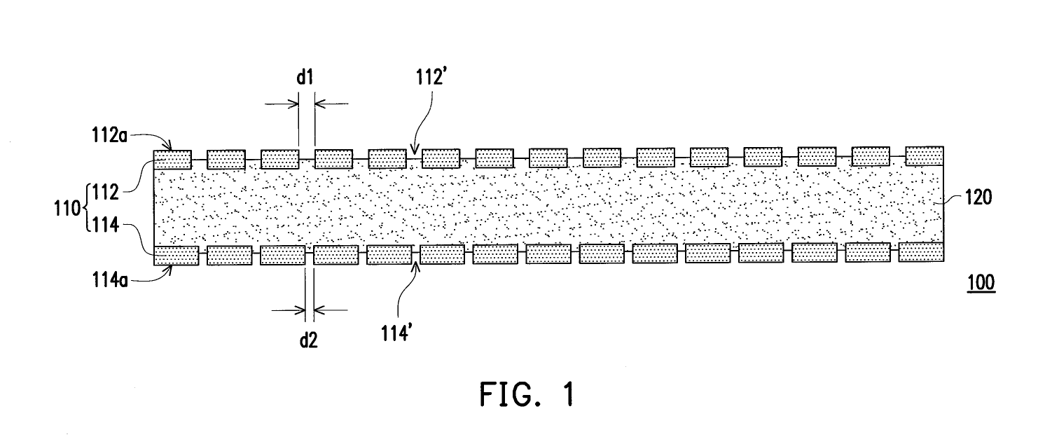

FIG. 1 is a schematic cross-sectional view of a dressing structure 100 according to a first embodiment of the invention. Please referring to FIG. 1, the dressing structure 100 includes a composite substrate layer 110 and a gel layer 120.

The composite substrate layer 110 includes a first substrate layer 112 and a second substrate layer 114. The first substrate layer 112 and the second substrate layer 114, for example, include porous membranes, and the porous membranes may be fabrics having an interweaving mesh structure and pores, or other suitable membrane structures. The material of the first substrate layer 112 or the material of the second substrate layer 114 may be composed of materials such as cotton, hemp, or chemical synthetic fiber. In particular, a material of the chemical synthetic fiber is, for example, polyethylene, polypropylene, polyester, polycarbonate, polyurethanes, a derivative thereof, or a combination thereof. In the present embodiment, the materials of the first substrate layer 112 and the second substrate layer 114 are the same, and the invention is not limited thereto. In other embodiments of the invention, the materials of the first substrate layer 112 and the second substrate layer 114 are different.

In addition, the first substrate layer 112 of the first embodiment includes a plurality of first pores 112′, and the second substrate layer 114 includes a plurality of second pores 114′, wherein a size d2 of the second pores 114′ is, for example, smaller than a size d1 of the first pores 112′. Besides, when using the dressing structure 100 of the present embodiment, a surface 112a of the first substrate layer 112 is in contact with wounds, and a surface 114a of the second substrate layer 114 is, for example, a outer surface exposed to the external. Because the size d2 of the second pores 114′ is smaller than that of the first pores 112′, it not only prevents permeation of the gel layer but also maintains good air permeability in the use of the dressing structure 100. Furthermore, the first substrate layer 112 and the second substrate layer 114 are flat and unwrinkled. Therefore, the design of the composite substrate layer 110 of the invention can avoid the defects, such as a poor product appearance, difficulties of being adhering evenly on the skin, discomfort, and so on, thereby enhancing the stability of the dressing structure 100 and improves the convenience and comfort in the use of the dressing.

In FIG. 1, the gel layer 120 is at least disposed between the first substrate layer 112 and the second substrate layer 114. The gel layer 120 includes, for example, a hydrogel layer or a polymer layer. It should be noted that, in the present embodiment, the gel layer 120 further exists inside the first pores 112′ of the first substrate layer 112, however the invention is not limited thereto. In the other embodiments of the invention, the gel layer 120 may also exist inside the second pores 114′ of the second substrate layer 114. Thus, since both of the first substrate layer 112 and the second substrate layer 114 have the pores, and the gel layer 120 can be embedded in the pores, which enhances the adhesion tension between the gel layer 120 and the first substrate layer 112 and the second substrate layer 114.

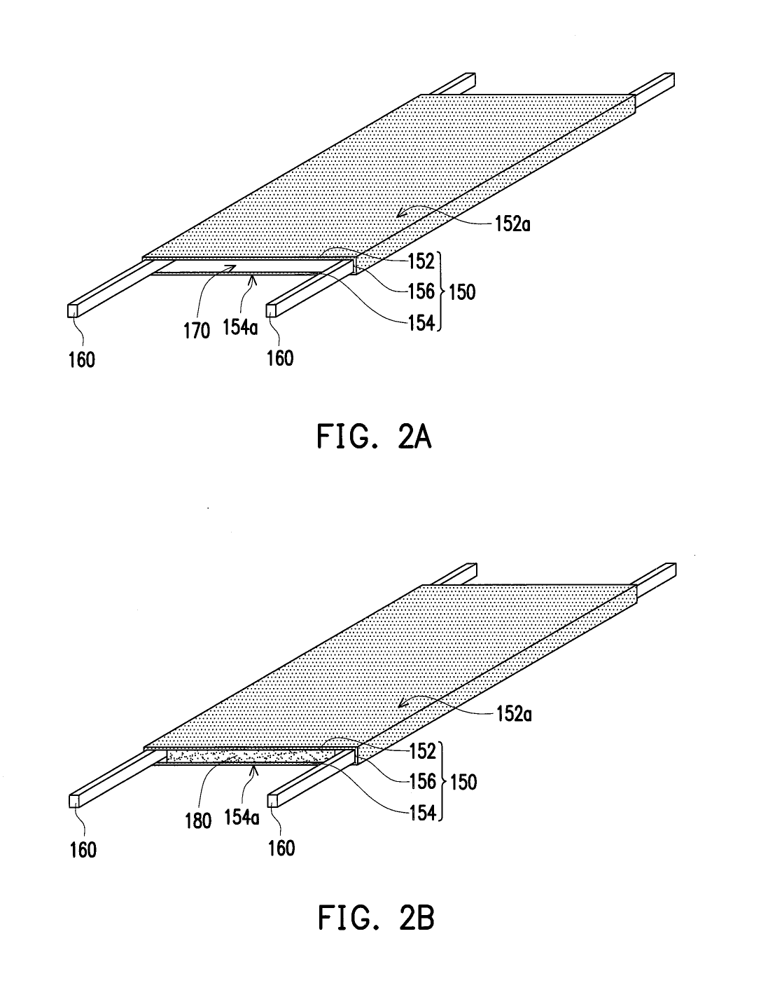

A fabrication process of the dressing structure 100 will be described in detail below. FIG. 2A through FIG. 2D are schematic perspective views of a fabrication process of the dressing structure 100 shown in FIG. 1.

First, referring to FIG. 2A, a composite substrate 150 is provided. The composite substrate 150 includes a first substrate portion 152, a second substrate portion 154, and side portions 156. The first substrate portion 152 and the second substrate portion 154 are porous membranes, and a pore size of the second substrate portion 154 (not illustrated) is, for example, smaller than a pore size of the first substrate portion 152 (not illustrated). In the present embodiment, the composite substrate 150 is exemplified by the first substrate portion 152, the second substrate portion 154 and the side portions 156, in which the first substrate portion 152, the second substrate portion 154 and the side portions 156 are integrally formed, but the invention is not limited thereto. In the other embodiments, the composite substrate 150 can be not integrally formed as well. For example, the first substrate portion 152 and the second substrate portion 154 are different materials respectively and adhered to each other by an adhesive (not shown). Moreover, the adhered region may be located on the side portions 156 connecting the first substrate portion 152 and the second substrate portion 154.

Next, a support device 160 is utilized to stretch the two side portions 156 of the composite substrate 150, hence the first substrate portion 152 and the second substrate portion 154 can be flat and unwrinkled, and a containing space 170 is formed between the first substrate portion 152 and the second substrate portion 154. In other words, the containing space 170 is defined as a space surrounded by the first substrate portion 152, the second substrate portion 154, and the support device 160.

Please referring to FIG. 2B, the unreacted primary material (not illustrated) is coated on a surface 152a of the first substrate portion 152. Since the primary material is usually a runny liquid and the first substrate portion 152 is a porous membrane, the primary material can permeate through the pores of the first substrate portion 152 to the containing space 170. Moreover, as the pores size of the second substrate portion 154 is smaller than that of the first substrate portion 152, the content of the unreacted primary material and the pore size of the second substrate portion 154 can be controlled to prevent the primary material located in the containing space 170 from permeating to a surface 154a of the second substrate portion 154. Next, the primary material is cured by photo-polymerization or thermal polymerization to form a gel material 180. Therefore, in the present embodiment, the support device 160 is utilized to stretch the two side portions 156 of the composite substrate 150, hence the composite substrate 150 (including the first substrate portion 152 and the second substrate portion 154) is still flat and unwrinkled after the gel material 180 is cured.

Please referring to FIG. 2C, the support device 160 is removed from the composite substrate 150. Next, the side portions 156 are cropped at least along with a trim line 190, so as to form the dressing structure 100 shown in FIG. 2D. In detail, the first substrate layer 112 and the second substrate layer 114 of the composite material layer 110 and the gel layer 120 in FIG. 2D are formed of the first substrate portion 152 and the second substrate portion 154 of the cropped composite substrate 150 and the gel material 180 respectively in FIG. 2C. In order to describe the fabrication process of the dressing structure 100 in detail, the pores (not illustrated) of the first substrate layer 112 and the second substrate layer 114 are omitted in the dressing structure 100 of FIG. 2D, and the detailed components of the dressing structure 100 can be referred to the description and the cross-sectional view of FIG. 1 for details.

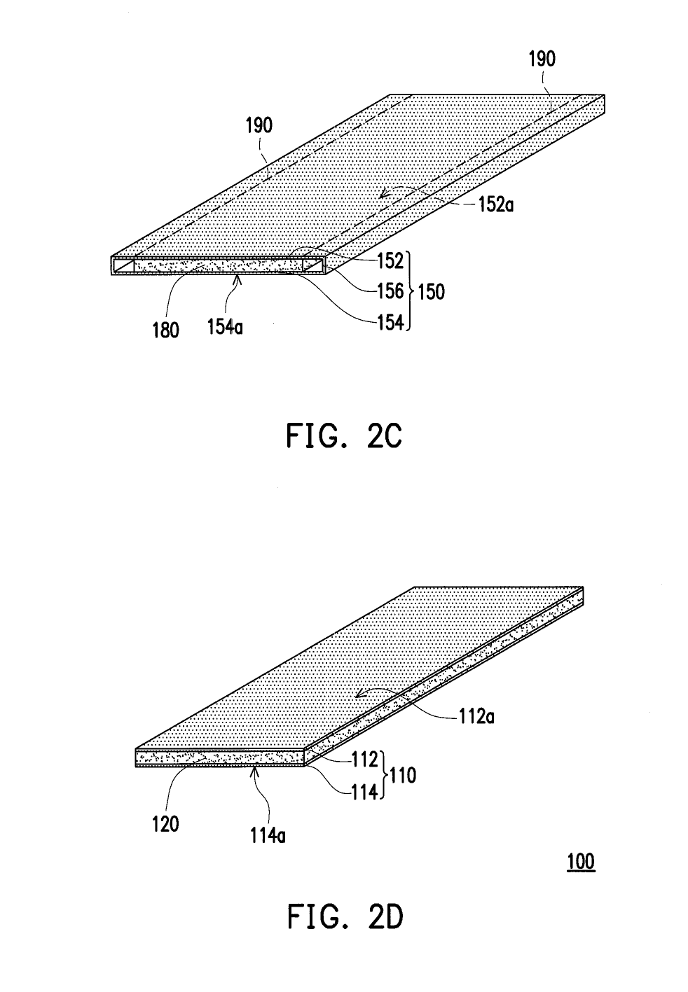

FIG. 3 is a schematic cross-sectional view of a dressing structure 200 according to a second embodiment of the invention. The embodiment of FIG. 3 is similar to the embodiment of FIG. 1, so the same devices are denoted by the same symbols, and descriptions thereof are omitted. Please referring to FIG. 3, one of differences between the embodiment of FIG. 3 and the embodiment of FIG. 1 is that a gel layer 220 further permeates through the pores 112′ of the first substrate layer 112 and then is disposed on the surface 112a of the first substrate layer 112. Namely, the gel layer 220 of the dressing structure 200 includes a first gel portion 222 and a second gel portion 224, in which the first gel portion 222 is located between the first substrate layer 112 and the second substrate layer 114, and the second gel portion 224 is located on the surface 112a of the first substrate layer 112. In detail, the pores 112′ of the first substrate layer 112 are filled up with the gel layer 220. Therefore, the gel layer 220 of the dressing structure 200 in the present embodiment does not only contact the skin with a larger area but also absorbs more wound exudates.

Due to the fabrication process of the dressing structure 200 is similar to the fabrication process of the dressing structure 100, only the differences are illustrated below. One of the differences is that the amount of demand unreacted primary material in the fabrication process of the dressing structure 200 is more than that of the dressing structure 100. Namely, the amount of unreacted primary material and the pore size of the second substrate portion 154 can be controlled, thereby the primary material is located between the first substrate layer 112 and the second substrate layer 124 and is further disposed on the surface 112a of the first substrate layer 112, which prevents the primary material from permeating to the surface 114a of the second substrate layer 114.

It should be noted that, in the present embodiment, the pore size d2 of the second pores 114′ is smaller than the pore size d1 of the first pores 112′, but the invention is not limited thereto. In other embodiments, the pore size d2 of the second pore 114′ may be equal to or larger than the pore size d1 of the first pore 112′, as long as the liquid (the unreacted primary material or the wound exudates) is prevented from permeating from the second substrate layer 114 to the surface 114a or the external. For instance, the third embodiment and the fourth embodiment are described below. Since a barrier layer is disposed on the surface 114a of the second substrate layer 114, which may prevent the liquid from permeating to the external, the size d2 of the second pores 114′ can be equal to or larger than the size d1 of the first pores 112′.

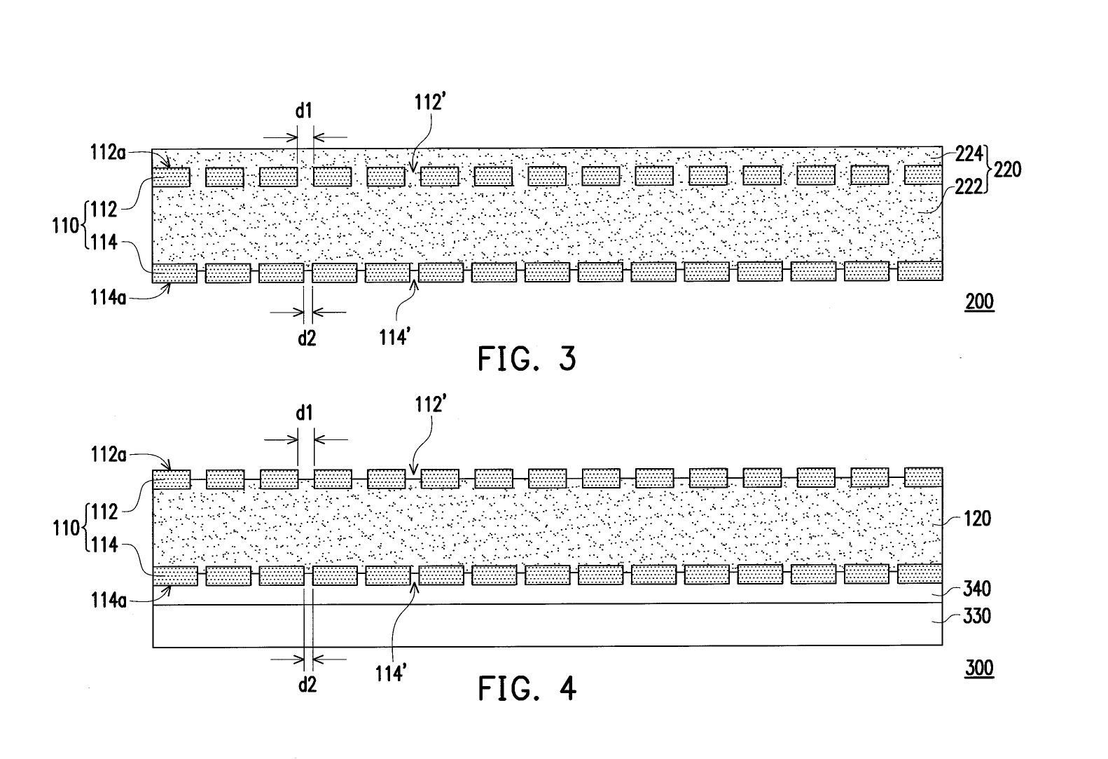

FIG. 4 is a schematic cross-sectional view of a dressing structure 300 according to a third embodiment of the invention. The embodiment of FIG. 4 is similar to the embodiment of FIG. 1, so the same devices are denoted by the same symbols, and descriptions thereof are omitted. Please referring to FIG. 4, one of differences between the embodiment of FIG. 4 and the embodiment of FIG. 1 is that the dressing structure 300 further includes a barrier layer 340 and a back layer 330.

The barrier layer 340 is disposed between the back layer 330 and the second substrate layer 114. The second pores 114′ of the surface 114a of the second substrate layer 114 can be covered or filled by the barrier layer 340 to prevent the liquid (the unreacted primary material or wound extudates, etc) from permeating from the second substrate layer 114 to a surface 330a of the back layer 330. The barrier layer 340 includes, for example, a non-porous membrane. In the present embodiment, the barrier layer 340 is, for example, a lamination layer, and the lamination layer is, for example, a polymer membrane directly formed on the second substrate layer 114 by lamination. The material of the polymer membrane includes polyethylene, polypropylene, polyester, polycarbonate, polyurethanes, a derivative thereof, or a combination thereof. In addition, since the barrier layer 340 substantially has a flat surface without pores, the contact area between the second substrate layer 114 and the back layer 330 is significantly increased, resulting in greater adhesion between the second substrate layer 114 and the back layer 330. In this way, the separation of the second substrate layer 114 from the back layer 330 during a process of replacing dressing, which is an inconvenient situation, may be avoided. Moreover, although the present embodiment illustrates one barrier layer 340, the invention is not limited thereto. In order words, in the other embodiments, the dressing structure 300 may also includes a plurality of stacked barrier layers 340, in which the barrier layer 340 may have the same or different materials.

The back layer 330 is disposed on the surface 114a of the second substrate layer 114 opposite to the gel layer 120. The back layer 330 has surfaces 330a and 330b. The surface 330a is, for example, an inner surface contacting the barrier layer 340, and the surface 330b is, for example, an outer surface exposed to the external. The main function of the back layer 330 is to provide water resistance for the dressing structure 300. In an embodiment, a layer of medical grade pressure sensitive adhesive (not illustrated) is, for example, coated on the surface 330a of the back layer 330 to fix the second substrate layer 114 on the back layer 330.

In the third embodiment above, the barrier layer 340 is directly formed on the second substrate layer 114, but the invention is not limited thereto. In other words, the barrier layer 340 may be joined directly to the second substrate layer 114 or joined to the second substrate layer 14 by layers such as an adhesive layer (as in the fourth embodiment below).

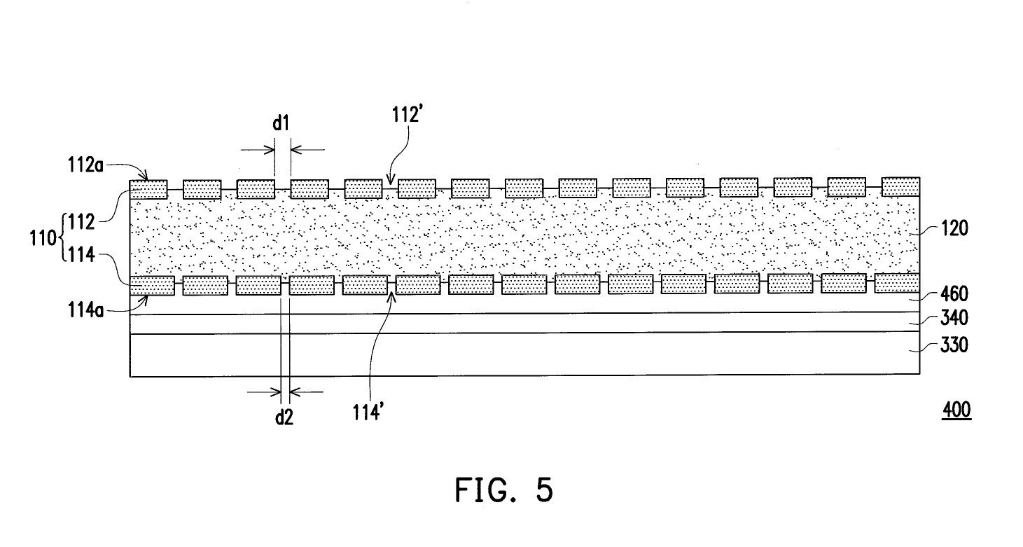

FIG. 5 is a schematic cross-sectional view of a dressing structure 400 according to a fourth embodiment of the invention. The embodiment of FIG. 5 is similar to the embodiment of FIG. 4, so the same devices are denoted by the same symbols, and descriptions thereof are omitted. Please referring to FIG. 5, one of differences between the embodiment of FIG. 5 and the embodiment of FIG. 1 is that the dressing structure 400 further includes an adhesive layer 460.

The adhesive layer 460 is disposed between the barrier layer 340 and the second substrate layer 114. The adhesive layer 460, for example, includes a hot melt adhesive, a pressure sensitive adhesive, or an acrylic glue. In detail, the adhesive layer 460 is coated on the barrier layer 340 after the barrier layer 340 is plastic molded or shaped, and the barrier layer 340 is joined to the second substrate layer 114 by the adhesive layer 460. Moreover, since the adhesive layer 460 substantially has a flat surface without pores, the contact area between the second substrate layer 114 and the barrier layer 340 is significantly increased, resulting in greater adhesion between the second substrate layer 114 and the barrier layer 340.

Based on above, the composite substrate layer in the dressing structure of the invention includes the first substrate layer and the second substrate layer, in which the second substrate layer and the first substrate layer are flat and unwrinkled. Therefore, the composite substrate layer according to the invention can avoid the defects, such as poor product appearance, uneven adhesion, discomfort, and so on, thereby enhancing the stability of the dressing and improves the convenience and comfort in the use of the dressing.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A dressing structure, comprising:
a composite substrate layer, comprising:
a first substrate layer, having a plurality of first pores; and
a second substrate layer, having a plurality of second pores, wherein the second substrate layer and the first substrate layer are flat and unwrinkled; and
a gel layer, disposed at least between the first substrate layer and the second substrate layer.

2. The dressing structure as claimed in claim 1, wherein the first substrate layer and the second substrate layer comprise porous membranes.

3. The dressing structure as claimed in claim 1, wherein the gel layer comprises a hydrogel layer or a polymer layer.

4. The dressing structure as claimed in claim 1, wherein the gel layer is further disposed in the first pores of the first substrate layer.

5. The dressing structure as claimed in claim 1, wherein the first pores of the first substrate layer are filled up with the gel layer such that the gel layer is further disposed on a surface of the first substrate layer.

6. The dressing structure as claimed in claim 1, wherein a size of the second pores is smaller than a size of the first pores.

7. The dressing structure as claimed in claim 1, wherein a size of the second pores is equal to a size of the first pores.

8. The dressing structure as claimed in claim 1, wherein a size of the second pores is larger than a size of the first pores.

9. The dressing structure as claimed in claim 1, further comprising a back layer disposed on a surface of the second substrate layer opposite to the gel layer.

10. The dressing structure as claimed in claim 9, further comprising a barrier layer disposed between the back layer and the second substrate layer.

11. The dressing structure as claimed in claim 10, wherein the barrier layer comprises a non-porous membrane.

12. The dressing structure as claimed in claim 11, wherein the barrier layer comprises a lamination layer.

13. The dressing structure as claimed in claim 11, wherein a material of the barrier layer comprises polyethylene, polypropylene, polyester, polycarbonate, polyurethane, a derivative thereof, or a combination thereof.

14. The dressing structure as claimed in claim 11, further comprising an adhesive layer disposed between the barrier layer and the second substrate layer.

15. The dressing structure as claimed in claim 14, wherein the adhesive layer comprises a hot melt adhesive, a pressure sensitive adhesive, or an acrylic glue.

16. The dressing structure as claimed in claim 1, wherein a material of the first substrate layer and a material of the second substrate layer independently comprise cotton, hemp, or chemical synthetic fiber.

17. The dressing structure as claimed in claim 16, wherein a material of the chemical synthetic fiber comprises polyethylene, polypropylene, polyester, polycarbonate, polyurethanes, a derivative thereof, or a combination thereof.

18. The dressing structure as claimed in claim 1, wherein the first substrate layer and the second substrate layer comprise the same materials.

19. The dressing structure as claimed in claim 1, wherein the first substrate layer and the second substrate layer comprise different materials.
